(19) United States
(12) Patent Application Publication
(10) Pub. No.: US 2010/0121020 A1
(43) Pub. Date: May 13, 2010

(54) 3.3'-Diamino-5,5'-Diphenyl-4,4'-Biphenyldiol and Raw Material Thereof as Well as Polybenzoxazole

(75) Inventors: Kenji Kunikata, Saitama (JP); Taihei Koumoto, Tokyo (JP); Kenji Sekine, Tokyo (JP)

Correspondence Address: PETERS VERNY , L.L.P., 425 SHERMAN AVENUE, SUITE 230, PALO ALTO, CA, 94306, US

(73) Assignee: NIPPONKAYAKU KABUSHIKIKAISHA, Tokyo (JP)

(21) Appl. No.: 12/531,600

(22) PCT Filed: Mar. 18, 2008
(86) PCT No.: PCT/JP2008/055011

§ 371 Date: Oct. 1, 2009

(30) Foreign Application Priority Data
Mar. 23, 2007 (JP) 2007/077475

(51) Int. Cl. C08G 73/22; C07C 205/23; C07C 215/80

(52) U.S. Cl. 528/289; 568/705; 564/443

(57) ABSTRACT

The invention provides 3,3′-diamino-5,5′-diphenyl-4,4′-biphenyldiol which is useful as a monomer for a high-performance polymer, particularly a polymer satisfying performances highly required in the semiconductor field such as polybenzoxazole, polyimide or the like and 3,3′-dinitro-5,5′-diphenyl-4,4′-biphenyldiol as a raw material thereof. Also, the invention provides a polybenzoxazole having a repeating unit represented by the following formula (1):

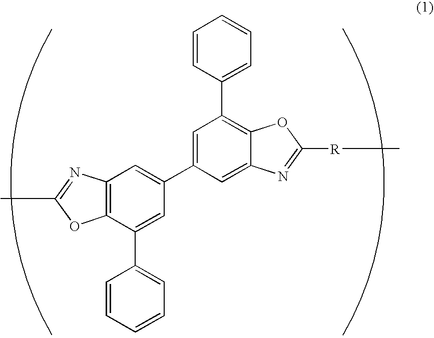

(wherein R is a bivalent dicarboxylic acid residue).

### TECHNICAL FIELD

This invention relates to 3,3′-diamino-5,5′-diphenyl-4,4′-biphenyldiol as a monomer useful for the production of high-performance polymers including polyimide, photosensitive polyimide, polybenzoxazole, photosensitive polybenzoxazole, polyhydroxyamide, polyhydroxyimide and polyamide as well as a raw material thereof. Also, this invention relates to a polybenzoxazole obtained by the reaction of 3,3′-diamino-5,5′-diphenyl-4,4′-biphenyldiol.

### BACKGROUND ART

Bis-o-aminophenol is a compound required for the production of a polymer having a high heat resistance, a high strength and excellent electric properties, for example, polybenzoxazole and polyimide having a hydroxyl group (polyhydroxyimide) or a precursor thereof. Polybenzoxazole and polyhydroxyimide are particularly important as a surface-protecting membrane of a semiconductor, an interlaminar insulating membrane, an interrelated insulating membrane of a multilayer printed wiring board, a cover coat of a flexible wiring board or the like. Recently, photosensitive polybenzoxazole and polyimide having a function as a photoresist are particularly watched. Polybenzoxazole is generally produced by making polyhydroxyamide from dicarboxylic dichloride and bis-o-aminophenol and then heat-treating it to conduct dehydration and ring closure. Also, polyhydroxyimide is generally produced by making polyamic acid from tetracarboxylic anhydride and bis-o-aminophenol and then heat-treating it to conduct dehydration and ring closure. Bis-o-aminophenol has a great influence on the properties of a polymer made therefrom. That is, the heat resistance, mechanical properties, electric properties, solubility and the like of the polymer are largely influenced by bis-o-aminophenol as a raw material. As a concrete example of bis-o-aminophenol used in such applications are known 2,2-bis(3-amino-4-hydroxyphenyl) propane, 2,2-bis(3-amino-4-hydroxyphenyl) hexafluoropropane, bis(3-amino-4-hydroxyphenyl) sulfone (see JP-A-2007-15967), 3,3′-diamino-4,4′-biphenyldiol (see JP-A-H11-106367), 3,3′-diamino-5,5-dimethyl-4,4′-biphenyldiol (see JP-A-H11-106367), 3,3′-diamino-5,5′-diphenylethenyl-4,4′-biphenyldiol (see JP-A-2005-97230) and so on.

### DISCLOSURE OF THE INVENTION

It is, therefore, an object of the invention to provide bis-o-aminophenol which is a monomer useful for the production of a polymer having excellent physical properties such as heat resistance, low expansibility, low dielectric constant, high reflection and the like in response to high densification, surface-packaging or high-frequency transmission increasingly proceeding in the semiconductor field.

The inventors have made various studies in order to solve the above problems and discovered that 3,3′-diamino-5,5′-diphenyl-4,4′-biphenyldiol is useful as a monomer for a polymer satisfying performances highly required in the semiconductor field as mentioned above and is produced by a method in a high purity and a high yield, and as a result the invention has been accomplished.

That is, the summary and construction of the invention are as follows.

1. 3,3′-Diamino-5,5′-diphenyl-4,4′-biphenyldiol.

2. 3,3′-Dinitro-5,5′-diphenyl-4,4′-biphenyldiol.

3. A polybenzoxazole having a repeating unit represented by the following formula (1):

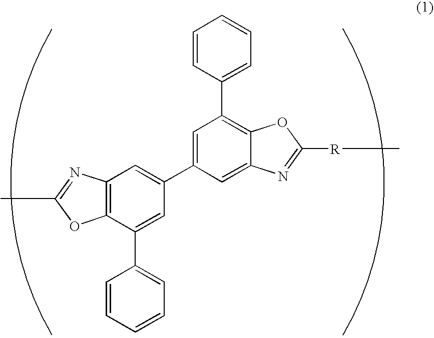

(wherein R is a bivalent dicarboxylic acid residue).

4. A polybenzoxazole according to the item 3, which is obtained by a polycondensation reaction of 3,3′-diamino-5,5′-diphenyl-4,4′-biphenyldiol with dicarboxylic acid or a derivative thereof.

3,3′-Diamino-5,5′-diphenyl-4,4′-biphenyldiol according to the invention is useful as a monomer for a high-performance polymer, particularly a polymer satisfying performances highly required in the semiconductor field.

### BEST MODE FOR CARRYING OUT THE INVENTION

### 3,3′-Diamino-5,5′-diphenyl-4,4′-biphenyldiol

The invention will be described in detail below. 3,3′-Diamino-5,5′-diphenyl-4,4′-biphenyldiol according to the invention is obtained, for example, by nitrating 3,3′-diphenyl-4,4′-biphenyldiol to obtain 3,3′-dinitro-5,5′-diphenyl-4,4′-biphenyldiol and then reducing 3,3′-dinitro-5,5′-diphenyl-4,4′-biphenyldiol. Moreover, 3,3′-diphenyl-4,4′-biphenyldiol as a raw material can be easily produced by an ordinary method starting from commercially available o-phenylphenol.

Also, 3,3′-dinitro-5,5′-diphenyl-4,4′-biphenyldiol according to the invention is a precursor substance of 3,3′-diamino-5,5′-diphenyl-4,4′-biphenyldiol as mentioned above, which is obtained, for example, by the nitration of 3,3′-diphenyl-4,4′-biphenyldiol. The nitration reaction is usually conducted by treating 3,3′-diphenyl-4,4′-biphenyldiol with nitric acid.

The nitric acid used for the nitration reaction is generally an aqueous solution having a concentration of 10-95% by mass, and industrial nitric acid may be used as it is or by diluting with water. A preferable concentration of nitric acid is 55-75% by mass. The amount of nitric acid (HNO₃) used is generally 1.8-3.8 moles, preferably 2.1-2.4 moles based on 1 mole of 3,3′-diphenyl-4,4′-biphenyldiol. Moreover, the reaction temperature in the nitration is generally −20 to 100° C., preferably −5 to 30° C.

The nitration of 3,3′-diphenyl-4,4′-biphenyldiol is conducted in a solvent inert to the reaction. As the usable inert solvent is mentioned an aliphatic acid, an aliphatic hydrocarbon, an aromatic hydrocarbon, a chlorinated aliphatic hydrocarbon or a chlorinated aromatic hydrocarbon. As a concrete example of the aliphatic acid may be mentioned acetic acid, propionic acid and the like. As a concrete example of the aliphatic hydrocarbon may be mentioned n-hexane, isohexane, n-heptane, n-octane, cyclopentane, cyclohexane, methylcyclohexane and the like. As a concrete example of the aromatic hydrocarbon may be mentioned benzene, toluene, xylene and the like. As a concrete example of the chlorinated aliphatic hydrocarbon may be mentioned dichloromethane, chloroform, carbon tetrachloride, 1,2-dichloroethane, 1,1,1-trichloroethane and the like. As a concrete example of the chlorinated aromatic hydrocarbon may be mentioned monochlorobenzene, ortho-dichlorobenzene and the like. The amount of the solvent used in the nitration reaction is usually 3-20 times of 3,3′-diphenyl-4,4′-biphenyldiol (mass ratio).

In detail, the nitration reaction is conducted by dispersing and dissolving 3,3′-diphenyl-4,4′-biphenyldiol in an inert solvent with stifling and adding a predetermined amount of nitric acid little by little thereto while holding at a predetermined temperature. The addition time of nitric acid is generally 0.5-20 hours, preferably 3-10 hours. After the addition of nitric acid, the stifling is further continued for 0.5-10 hours while holding at the predetermined temperature to complete the reaction. After the completion of the reaction, the reaction mixture is added with water while stifling, and thereafter the reaction mixture is filtrated in case that it is in the form of slurry and well washed with water, whereby there can be obtained a crude nitro-body. The status of the reaction and the purity of the target substance can be confirmed by a high performance liquid chromatography (HPLC). The thus obtained crude nitro-body can be purified with an organic solvent such as dimethylformamide, dimethylacetamide, N-methylpyrrolidone, methanol, ethanol, isopropanol, acetone, methyl ethyl ketone, methoxyethanol, dioxane, toluene, ethyl acetate or the like through hot washing, recrystallization and so on.

The thus obtained 3,3′-dinitro-5,5′-diphenyl-4,4′-biphenyldiol according to the invention can be reduced to obtain 3,3′-diamino-5,5′-diphenyl-4,4′-biphenyldiol. As a preferable embodiment of reduction reaction will be described a method of reducing with hydrogen or hydrazines in the presence of a catalyst below.

As the catalyst usable for the reduction reaction are mentioned a nickel catalyst, a noble metal catalyst and so on. As the nickel catalyst may be used nickel dispersed on a carrier such as diatomaceous earth, active carbon or the like, nickel boride, Raney nickel and so on. Among these nickel catalysts, Raney nickel is preferable. As the noble metal catalyst may be used a platinum catalyst, a palladium catalyst, a ruthenium catalyst, a rhodium catalyst and so on. As the noble metal catalyst may be used a single element of a noble metal as well as an oxide of the noble metal, a catalyst of the noble metal dispersed on a carrier such as silica, alumina, zeolite, calcium carbonate or the like, and so on. Among these noble metal catalysts, a catalyst of platinum or palladium dispersed on active carbon is preferable. When the catalyst of platinum or palladium dispersed on active carbon is used, a proportion of noble metal to be dispersed on the carrier is preferably 1.0-20% by mass. Also, in case of reducing with hydrazines, a combination of an iron salt and active carbon may be used as a catalyst. As the iron salt may be mentioned ferrous chloride, ferric chloride and the like. The amount of the catalyst used is preferable to be 5-20% by mass in case of Raney nickel, 1-10% by mass in case of platinum-carbon or palladium-carbon and 5-20% by mass in case of iron salt based on mass of 3,3′-dinitro-5,5′-diphenyl-4,4′-biphenyldiol as the raw material, respectively. The catalyst usable in the reduction reaction may be a new catalyst or a recovered and recycled catalyst.

When hydrogen is used in the reduction reaction, the reaction temperature is generally 30-150° C., preferably 50-100° C. When the reaction temperature is lower than 30° C., the reaction rate is late, while when it exceeds 150° C., a by-product due to the hydrogenation of aromatic nuclear or the like increases. The hydrogen pressure for reaction is generally 1-100 kg/cm² (gage pressure), preferably 2-20 kg/cm² (gage pressure). The hydrogen pressure for reaction is preferably held at a certain pressure by properly replenishing hydrogen, and also the replenishing of hydrogen is preferably conducted until hydrogen absorption stops. The amount of hydrogen absorbed is about 6.0 moles based on 1.0 mole of 3,3′-dinitro-5,5′-diphenyl-4,4′-biphenyldiol.

On the other hand, when hydrazines are used, as the hydrazines may be used hydrazine monohydrate, methylhydrazine, hydrazinium chloride, hydrazinium sulfate and so on. Among them, hydrazine monohydrate is preferable and, for example, it is used as an aqueous solution of 40-80% by mass of hydrazine monohydrate. The amount of hydrazines used is generally 2.5-10.0 moles, preferably 3.0-4.5 moles based on 1.0 mole of 3,3′-dinitro-5,5′-diphenyl-4,4′-biphenyldiol. The reaction temperature is generally 10-100° C., preferably 30-80° C.

The reduction reaction is generally conducted in a solvent such as lower aliphatic alcohols, cyclic or acyclic ethers, N-methylpyrrolidone or the like. As a concrete example of the usable lower aliphatic alcohols are mentioned methanol, ethanol, n-propanol, isopropanol, n-butanol, sec-butanol and so on. As a concrete example of the usable cyclic or acyclic ethers are mentioned ethylene glycol monomethyl ether, ethylene glycol monoethyl ether, ethylene glycol diethyl ether, dioxane, tetrahydrofuran and so on. The amount of the solvent used in the reduction reaction is generally 0.5-30 parts by mass, preferably 1-15 parts by mass based on 1 part by mass of 3,3′-dinitro-5,5′-diphenyl-4,4′-biphenyldiol. Also, a mixed solvent of these solvents and water may be used. The proportion of water in the mixed solvent is preferable to be not more than 50% by volume. A particularly preferable solvent in the reduction reaction is a mixed solvent of N-methylpyrrolidone and methanol. The mixing ratio of N-methylpyrrolidone/methanol is generally 10/90-90/10, preferably 20/80-80/20, more preferably 30/70-70/30, particular preferably 40/60-60/40 as a volume ratio.

As a reduction reaction using hydrogen is concretely mentioned a method wherein predetermined amounts of a solvent, 3,3-dinitro-5,5′-diphenyl-4,4′-biphenyldiol and a catalyst are charged into an autoclave and air in the autoclave is replaced with nitrogen and then hydrogen. In this reduction reaction, the reaction mixture is heated close to a reaction temperature with stirring to start the reaction. Since heat generation occurs with the progress of the reduction reaction, the reaction temperature is maintained by cooling and hydrogen is properly replenished under a given hydrogen pressure for reaction until hydrogen absorption stops, whereby the reduction reaction is conducted. Also, there may be a method in which a solvent and a catalyst are charged into an autoclave and then a mixture of 3,3′-dinitro-5,5′-diphenyl-4,4′-biphenyldiol and a solvent is supplied together with hydrogen while holding the reaction temperature and pressure, whereby the reaction is conducted. The reaction time varies depending on an amount of catalyst, a reaction temperature and a reaction pressure, but is generally 0.5-12 hours. After the completion of the reaction, the autoclave is cooled down to room temperature and hydrogen is replaced with nitrogen, and thereafter the catalyst is filtered off from the reaction mixture taken out from the autoclave. The solvent and water are distilled off from the filtrate to obtain solid 3,3′-diamino-5,5′-diphenyl-4,4′-biphenyldiol.

As a reduction reaction using hydrazines is concretely mentioned a method wherein air in a reaction vessel is replaced with nitrogen and thereafter predetermined amounts of a solvent, 3,3′-dinitro-5,5′-diphenyl-4,4′-biphenyldiol and a catalyst are charged into the reaction vessel and heated close to a reaction temperature with stirring and then an aqueous solution of hydrazines is added to conduct the reaction. Since heat generation occurs with the progress of the reduction reaction, the aqueous solution of a predetermined amount of hydrazines is added separately or continuously to conduct the reduction reaction while maintaining the reaction temperature by cooling. Also, there may be a method in which a solvent and a catalyst are charged into a reaction vessel purged with nitrogen and then a reaction solvent slurry or solution of 3,3′-dinitro-5,5′-diphenyl-4,4′-biphenyldiol is added together with an aqueous solution of hydrazines to conduct the reaction while maintaining the reaction temperature. The reaction time varies depending on an amount of catalyst and a reaction temperature, but is generally 0.5-12 hours. After the addition of hydrazines, the stifling is further continued for 0.5-6 hours while maintaining the predetermined reaction temperature. After the completion of the reaction, the reaction mixture is taken out after the cooling to room temperature and the catalyst is removed by filtration. The solvent and water are distilled off from the filtrate to obtain solid 3,3′-diamino-5,5′-diphenyl-4,4′-biphenyldiol.

Also, when the mixed solvent of N-methylpyrrolidone and methanol is used as a reaction solvent in the reduction using hydrogen or hydrazines, a filtrate obtained by removing the catalyst from the reaction mixture through filtration after the completion of the reaction is added dropwise to an aqueous solution of hydrazine monohydrate under a nitrogen gas stream to separate out a crystal of 3,3′-diamino-5,5′-diphenyl-4,4′-biphenyldiol, which may be filtered and washed with water to obtain 3,3′-diamino-5,5′-diphenyl-4,4′-biphenyldiol. The concentration of hydrazine monohydrate used in the aqueous solution of hydrazine monohydrate is generally 0.05-20% by mass, preferably 0.5-15% by mass, more preferably 1-10% by mass. Moreover, the aqueous solution of hydrazine monohydrate used herein may include alcohols such as methanol, ethanol, isopropanol and the like and ethers such as ethylene glycol monoethyl ether, dioxane, tetrahydrofuran and the like. Also, the filtrate obtained by removing the catalyst from the reaction mixture is added with methanol under the nitrogen gas stream to separate out the crystal of 3,3′-diamino-5,5′-diphenyl-4,4′-biphenyldiol, and further water or the aqueous solution of hydrazine monohydrate is added dropwise to complete the separating of the crystal, which can be filtered and washed with water to obtain 3,3′-diamino-5,5′-diphenyl-4,4′-biphenyldiol.

Moreover, the reaction status in the reduction reaction or the purity of target substance can be confirmed by a high performance liquid chromatography (HPLC).

3,3′-Diamino-5,5′-diphenyl-4,4′-biphenyldiol according to the invention is obtained, for example, by conducting the aforementioned nitration and reduction reactions. The purity of 3,3′-diamino-5,5′-diphenyl-4,4′-biphenyldiol obtained by such reactions is sufficiently high, which can be further improved by purifying through recrystallization method or the like, if necessary. As a solvent used in the recrystallization method are mentioned, for example, alcohols such as methanol, ethanol, isopropanol and the like, ethers such as ethylene glycol monoethyl ether, dioxane, tetrahydrofuran and the like, aromatic hydrocarbons such as toluene, xylene and the like, and carboxylic acid ester such as methyl acetate, ethyl acetate and the like. In this case, the solvent is generally used in an amount of 1-10 parts by mass based on 1 part by mass of 3,3′-diamino-5,5′-diphenyl-4,4′-biphenyldiol. Moreover, these solvents may be used alone or in a combination of two or more.

In addition to the recrystallization method, the purification may be also conducted by a method wherein 3,3′-diamino-5,5′-diphenyl-4,4′-biphenyldiol is dissolved in diluted hydrochloric acid and added with active carbon with stifling and thereafter the active carbon is filtered off and the resulting filtrate is neutralized by adding an aqueous alkali solution such as caustic soda or the like to separate out 3,3′-diamino-5,5′-diphenyl-4,4′-biphenyldiol. In this case, 2-20 parts by mass of diluted hydrochloric acid is preferably used based on one part by mass of 3,3′-diamino-5,5′-diphenyl-4,4′-biphenyldiol. Moreover, the concentration of the diluted hydrochloric acid and the amount used thereof are preferably adjusted so that an amount of hydrogen chloride based on one mole of 3,3′-diamino-5,5′-diphenyl-4,4′-biphenyldiol is about 4-5 moles.

3,3′-Diamino-5,5′-diphenyl-4,4′-biphenyldiol according to the invention has two amino groups and two phenolic hydroxyl groups, and hence it can be used as a precursor material for polyimide resin by applying techniques described, for example, in JP-A-558-322929, WO 2003/060010 and so on. Also, 3,3′-diamino-5,5′-diphenyl-4,4′-biphenyldiol according to the invention can be used as a precursor material for polybenzoxazole by applying techniques described, for example, in JP-A-H11-322929, JP-A-2003-292620, JP-A-2006-45321, JP-A-2006-265384 and so on in addition to techniques described in detail below.

### Polybenzoxazole

Polybenzoxazole according to the invention is a polybenzoxazole having a repeating unit represented by the above formula (1), which is obtained by polycondensating 3,3′-diamino-5,5′-diphenyl-4,4′-biphenyldiol with dicarboxylic acid or a derivative thereof, for example, in the presence of a condensation agent. Moreover, 3,3′-diamino-5,5′-diphenyl-4,4′-biphenyldiol is produced by the above-mentioned method.

In the formula (1), R represents a residue formed by reacting dicarboxylic acid or the derivative thereof with 3,3′-diamino-5,5′-diphenyl-4,4′-biphenyldiol through dehydration and cyclization, i.e., a bivalent dicarboxylic acid residue.

For example, an alicyclic dicarboxylic acid having an alicyclic structure in its molecule is mentioned as the dicarboxylic acid. As the alicyclic dicarboxylic acid are concretely mentioned a cycloalkyl dicarboxylic acid having a carbon number of 3-8 such as 1,2-cyclohexane dicarboxylic acid, 1,3-cyclohexane dicarboxylic acid, 1,4-cyclohexane dicarboxylic acid, 1,1-cyclopropane dicarboxylic acid, 1,1-cyclobutane dicarboxylic acid, 1,3-cyclopentane dicarboxylic acid or the like; 2,5-norbornane dicarboxylic acid, 1,3-adamantane dicarboxylic acid and so on. Among them, the cycloalkyl dicarboxylic acid having a carbon number of 3-8 is preferable, and 1,3-cyclohexane dicarboxylic acid and 1,4-cyclohexane dicarboxylic acid are more preferable, and 1,3-cyclohexane dicarboxylic acid is particularly preferable from a viewpoint of the polymerization reactivity of polybenzoxazole and the solubility thereof to an organic solvent. Also, dicarboxylic acid other than the alicyclic dicarboxylic acid may be used as the dicarboxylic acid. As the dicarboxylic acid other than alicyclic dicarboxylic acid are mentioned, for example, terephthalic acid, isophthalic acid, phthalic acid, 2,5-dimethylterephthalic acid, 2,3-pyridine dicarboxylic acid, 2,4-pyridine dicarboxylic acid, 2,6-pyridine dicarboxylic acid, 3,4-pyridine dicarboxylic acid, 3,5-pyridine dicarboxylic acid, 4,4′-biphenyldicarboxylic acid, 2,2′-biphenyldicarboxylic acid, 4,4′-diphenyl ether dicarboxylic acid, 4,4′-diphenyl methane dicarboxylic acid, 4,4′-diphenyl sulfone dicarboxylic acid, 1,2-naphthalene dicarboxylic acid, 1,4-naphthalene dicarboxylic acid, 1,5-naphthalene dicarboxylic acid, 2,3-naphthalene dicarboxylic acid, 2,6-naphthalene dicarboxylic acid, 2,7-naphthalene dicarboxylic acid, 1,8-anthracene dicarboxylic acid, oxalic acid, malonic acid, succinic acid, glutaric acid, adipic acid, pimelic acid, spelic acid, azelaic acid, sebacic acid, maleic acid, fumaric acid and so on. As the derivative of dicarboxylic acid are mentioned acid halide and acid anhydride of the above dicarboxylic acid and so on. Moreover, these dicarboxylic acids and derivatives thereof may be used alone or in a combination of two or more.

A preferable method of producing the polybenzoxazole according to the invention is described in detail below. The polybenzoxazole according to the invention is obtained by polycondensating 3,3′-diamino-5,5′-diphenyl-4,4′-biphenyldiol with dicarboxylic acid or a derivative thereof, for example, in the presence of a condensation agent as mentioned above. Concretely, dicarboxylic acid (including the derivative of dicarboxylic acid, the same shall apply hereinafter) and 3,3′-diamino-5,5′-diphenyl-4,4′-biphenyldiol are charged in equal molar quantity into a reaction vessel and a polymerization solvent is added to the reaction vessel. Then, the resulting mixture is heated stepwise from 100° C. up to a final temperature every 10° C. in a nitrogen atmosphere (which is held at each temperature for 10 minutes) and finally held at 200-230° C. for 10 minutes to 2 hours while stirring with an agitator. In this way, the polycondensation reaction of dicarboxylic acid and 3,3′-diamino-5,5′-diphenyl-4,4′-biphenyldiol is conducted. Then, white precipitates of polybenzoxazole can be obtained by cooling the reaction product to room temperature, precipitating in water, washing with a large amount of water until a washing water neutralizes, further washing with methanol and finally drying at 100° C. under vacuum.

In the polycondensation reaction, the concentration in total of monomers is generally 5-30% by mass, preferably 7-20% by mass. When the concentration in total of monomers is less than 5% by mass, there is a tendency that the polymerization degree of polybenzoxazole is not increased sufficiently, while when it exceeds 30% by mass, the monomers may be not dissolved sufficiently to obtain a homogenous solution.

Also, the polymerization solvent and the condensation agent are not particularly limited. As the polymerization solvent possessing the condensation agent is preferably mentioned polyphosphoric acid or phosphorus pentoxide-methane sulfonic acid mixture.

In the polycondensation reaction, the temperature is preferable to be raised to at least 200° C. When the polycondensation reaction is conducted at a temperature of lower than 200° C., there is a fear that the polymerization degree is not increased sufficiently. While, the polymerization temperature is preferably achieved though the gradual rising as mentioned above, and hence it should not be raised violently, for example, up to 200° C. Otherwise, the alicyclic structure is partially decomposed to significantly stain the finally obtained polybenzoxazole and further the polymerization degree may not be increased sufficiently.

In the polybenzoxazole according to the invention, as a bis-o-aminophenol compound in addition to 3,3′-diamino-5,5′-diphenyl-4,4′-biphenyldiol can be properly selected and used 2,2-bis(3-amino-4-hydroxyphenyl)hexafluoropropane, 4,6-diamino resorcinol, 2,5-diamino hydroquinone, 3,3′-dihydroxy benzidine, 3,3′-diamino-4,4′-dihydroxybiphenyl, 4,4′-diamino-3,3′-dihydroxybiphenyl ether, 3,3′-diamino-4,4′-dihydroxybiphenyl sulfone, 4,4′-diamino-3,3′-dihydroxybiphenyl sulfone, 3,3′-diamino-4,4′-dihydroxybiphenyl methane, 4,4′-diamino-3,3′-dihydroxybiphenyl methane, 2,2-bis(3-amino-4-hydroxyphenyl) propane and so on within a scope of not damaging the demand characteristics and polymerization reactivity of the polybenzoxazole according to the invention. Moreover, these bis-o-aminophenol compounds may be used alone or in a combination of two or more. Also, the proportion of bis-o-aminophenol compound other than 3,3′-diamino-5,5′-diphenyl-4,4′-biphenyldiol in total of bis-o-aminophenol compounds is preferable to be not more than 40 mole %.

The polybenzoxazole according to the invention can be also synthesized through a precursor thereof, i.e., polyhydroxyamide or silylated polyhydroxyamide. For example, polyhydroxyamide is obtained by the polycondensation reaction of 3,3′-diamino-5,5′-diphenyl-4,4′-biphenyldiol and dicarboxylic dichloride. On the other hand, silylated polyhydroxyamide can be obtained, for example, as a high polymer by converting 3,3′-diamino-5,5′-diphenyl-4,4′-biphenyldiol with a silylation agent in an aprotic organic solvent such as N-methyl-2-pyrrolidone or the like into tetrasilylated form and then polycondensating with dicarboxylic dichloride in equal moles.

### EXAMPLES

The invention will be concretely described below with reference to examples, but is not limited thereto.

### Example 1

### Production of 3,3′-dinitro-5,5′-diphenyl-4,4′-biphenyldiol

Into a 1 L four-neck flask equipped with a thermometer and a stirrer are charged 510 ml of acetic acid and 50.7 g of 3,3′-diphenyl-4,4′-biphenyldiol (0.15 mol), and then 32.4 g (0.36 mol) of 70 mass % nitric acid (specific gravity d=1.42) is added dropwise over 3 hours while maintaining the reaction temperature at 18-25° C. in a water bath. The stirring is further continued for 4 hours while maintaining the reaction temperature to complete nitration reaction. The reaction mixture is added with 50 ml of water and heated at about 50° C. for 1 hour. This reaction mixture is cooled to room temperature and then filtered, and the resulting residue is sufficiently washed with water. The residue is dried under a reduced pressure to obtain 53.7 g of yellow-brown crude 3,3′-dinitro-5,5′-diphenyl-4,4′-biphenyldiol. The yield is 83.7% based on a theoretical yield. Moreover, the purity of 3,3′-dinitro-5,5′-diphenyl-4,4′-biphenyldiol through HPLC analysis is 93.7% (area %).

Then, 53.7 g of 3,3′-dinitro-5,5′-diphenyl-4,4′-biphenyldiol obtained is added to 215 g of dimethylformamide, heated to 70-76° C., stirred for 2 hours and then cooled to 30° C. and filtered. The filter cake is washed with methanol and dried to obtain 46.7 g of 3,3′-dinitro-5,5′-diphenyl-4,4′-biphenyldiol purified. The purified yield is 87% and the purity of target substance through HPLC analysis is 99.3% (area %).

The characteristic values of 3,3′-dinitro-5,5′-diphenyl-4,4′-biphenyldiol purified are as follows.

Melting point (DSC): 263.1° C.

¹H-NMR (CDCl₃): δ 7.46-7.51 ppm (2H), 7.51-7.58 (4H), 7.60-7.63 (4H), 7.89 (2H), 8.37 (2H) 11.16 (2H)

### Example 2

### Production of 3,3′-diamino-5,5′-diphenyl-4,4′-biphenyldiol (hydrogen reduction method)

Into a 500 cc autoclave equipped with a thermometer and a stirrer are charged 40.0 g (0.0934 mol) of 3,3′-dinitro-5,5′-diphenyl-4,4′-biphenyldiol obtained in Example 1, 168 ml of methanol and 72 ml of N-methylpyrrolidone and further 1.8 g (as expressed by weight as a dried product) of 5% Pd-carbon, and thereafter the interior of the vessel is replaced with nitrogen and then hydrogen. When the reduction reaction is conducted while holding at a hydrogen pressure of 9 kg/cm² (gage pressure) and 60° C., hydrogen absorption stops in about 2 hours. Further, after aging at 60° C. for 30 minutes, the interior of the vessel is replaced with nitrogen to take out the reaction mixture from the autoclave. After the catalyst is removed from the reaction mixture through filtration, the resulting filtrate is added dropwise at room temperature under a nitrogen gas stream to an aqueous solution previously prepared with 18.7 g of an aqueous solution of 60% by mass of hydrazine monohydrate and 400 ml of water, stirred for 30 minutes, cooled to 2-3° C. and then filtered. The resulting residue is dried under a reduced pressure to obtain 32.7 g of white 3,3′-diamino-5,5′-diphenyl-4,4′-biphenyldiol. The yield is 95%. Also, the purity of target substance through HPLC analysis is 99.5% (area %).

The characteristic values of 3,3′-diamino-5,5′-diphenyl-4,4′-biphenyldiol obtained are as follows.

Melting point (DSC): 107.8° C.

¹H-NMR (CDCl₃): δ 3.7-4.0 ppm (4H), 5.1-5.4 (2H), 6.88 (2H), 6.95 (2H), 7.35-7.45 (2H), 7.45-7.60 (8H)

### Example 3

### Production of 3,3′-diamino-5,5′-diphenyl-4,4′-biphenyldiol (hydrazine reduction method)

Into a 500 ml flask equipped with a thermometer, a stirrer and a condenser are charged 40 g (0.0934 mol) of 3,3′-dinitro-5,5′-diphenyl-4,4′-biphenyldiol obtained in Example 1, 250 ml of methanol, 100 ml of N-methylpyrrolidone and 1.3 g of 10% Pd-carbon (active carbon, NNA manufactured by CHEMCAT CORPORATION) (dry mass), and the interior of the vessel is replaced with nitrogen. After the heating to 60° C., 31.0 g of an aqueous solution of 60% by mass of hydrazine monohydrate (0.3715 mol) is added dropwise over 4 hours, during which the cooling is conducted while maintaining the reaction temperature at 60-70° C. Further, after aging at 68-70° C. for 3 hours, the reaction mixture is cooled to room temperature. It is taken out, and a filtrate obtained by removing the catalyst from the reaction mixture through filtration is treated in the same manner as in Example 2 to obtain 33.7 g of 3,3′-diamino-5,5′-diphenyl-4,4′-biphenyldiol. The yield is 98%. Also, the purity of target substance through HPLC analysis is 99.2% (area %). The melting point and ¹H-NMR data of 3,3′-diamino-5,5′-diphenyl-4,4′-biphenyldiol obtained are completely coincident with the data described in Example 2.

### Example 4

### Production of Polybenzoxazole

### Synthesis of Precursor of Polybenzoxazole

Into a five-neck flask equipped with a stirrer, a reflux condenser and a thermometer is charged 3.22 g (0.01 mol) of 3,3′-diamino-5,5′-diphenyl-4,4′-biphenyldiol obtained in Example 2, which is dissolved by adding 9.37 g of N-methyl-2-pyrrolidone and 2.77 g of pyridine. To this solution is added dropwise a solution prepared by dissolving 1.78 g (0.01 mol) of isophthaloyl chloride in 12.5 g of N-methyl-2-pyrrolidone at −10° C. to 0° C. under a nitrogen gas stream and then stirred at room temperature for 3 hours. The resulting reaction solution is added dropwise to 300 g of methanol, and the resulting precipitates are dried to obtain a precursor of polybenzoxazole. The weight-average molecular weight (Mw) of the resulting polybenzoxazole precursor as converted to polystyrene is 14,000 as measured by a gel permeation chromatography (GPC) manufactured by TOSOH CORPORATION. As the weight reduction of the polybenzoxazole precursor is measured by using a thermogravimetric analyzer (TG-DTA) manufactured by SEIKO CORPORATION in a nitrogen atmosphere in a temperature-rising process at a temperature-rising rate of 10° C./minute, the weight loss due to thermal ring-closing is observed at 220-400° C.

### Synthesis of Polybenzoxazole

The thus obtained precursor of polybenzoxazole is subjected to a thermal ring-closing treatment under a nitrogen atmosphere at 350° C. for 3 hours to obtain polybenzoxazole. The weight loss of the resulting polybenzoxazole is measured by using DSC in a nitrogen atmosphere at a temperature-rising rate of 10° C./minute. The temperature of 5% weight loss is 610° C.

1. 3,3′-Diamino-5,5′-diphenyl-4,4′-biphenyldiol.

2. 3,3′-Dinitro-5,5′-diphenyl-4,4′-biphenyldiol.

3. A polybenzoxazole having a repeating unit represented by the following formula (1): 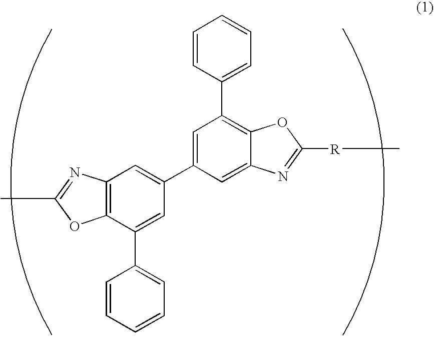
(wherein R is a bivalent dicarboxylic acid residue).

4. A polybenzoxazole according to claim 3, which is obtained by polycondensating 3,3′-diamino-5,5′-diphenyl-4,4′-biphenyldiol with dicarboxylic acid or a derivative thereof.
